(19) United States
(12) Patent Application Publication
(10) Pub. No.: US 2015/0182439 A1
(43) Pub. Date: Jul. 2, 2015

(54) SUNSCREENS

(71) Applicants: Christine Mendrok-Edinger, Basel (CH); Horst Westenfelder, Basel (CH)

(72) Inventors: Christine Mendrok-Edinger, Basel (CH); Horst Westenfelder, Basel (CH)

(73) Assignee: DSM IP ASSETS B.V., Heerlen (NL)

(21) Appl. No.: 14/124,681

(22) PCT Filed: Jun. 6, 2012
(86) PCT No.: PCT/EP2012/060665

§ 371 Date: May 6, 2014

(30) Foreign Application Priority Data
Jun. 8, 2011 (EP) 11169200.0
Jun. 15, 2011 (EP) 11169923.7

(51) Int. Cl. A61K 8/49; A61K 8/896; A61K 8/86; A61Q 17/04; A61K 8/29

(52) U.S. Cl. CPC A61K 8/496; A61Q 17/04; A61K 8/29; A61K 8/86; A61K 8/896

(57) ABSTRACT

The present invention relates to a composition for topical application comprising in a physiologically acceptable medium at least a benzotriazol derivative, a microfine metal oxide particle and a polymeric emulsifier. More specifically, the invention relates to compositions that exhibit a reduced whitening effect on the skin.

The present invention relates to a composition for topical application comprising in a physiologically acceptable medium at least a benzotriazol derivative, a microfine metal oxide particle and a polymeric emulsifier. More specifically, the invention relates to compositions that exhibit a reduced whitening effect on the skin.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancer. Thus, today's focus is towards eliminating as much of UVA (320-400 nm) and/or UVB (280-320 nm) light as possible. Consequently, there's a constantly increasing need for sun care products exhibiting high SPF's (Sun Protection Factor) and high UVA protection while being photostable.

In addition to chemical UV-filter substances, the use of physical UV-filter substances is known in the art. Such physical UV-filter substances are e.g. microfine zinc oxide and microfine titanium dioxide which are suspended in the respective cream or lotion and which are highly appreciated because of their excellent safety profile. However, such physical UV-filter substances have significant disadvantages. They in general exhibit lower effectiveness in absorbing UV radiation as compared to many chemical UV-filter substances. When their concentration is increased to promote greater effectiveness, an undesirable white layer appears on the skin which most users find objectionable.

It was therefore the object of the present invention to remedy the disadvantages of the prior art and to develop sun care products comprising microfine metal oxide particles which exhibit a reduced skin whitening effect.

Surprisingly, it has been found that the use of a polymeric emulsifier in the presence of specific benzotriazol derivatives reduces the whitening effect of the microfine metal oxide particles. Therefore, the metal oxide particles can be added to topical compositions at elevated blend ratios resulting in higher SPF's.

Thus, the invention relates in one aspect to a topical composition comprising a polymeric emulsifier, a microfine metal oxide particle and at least one benzotriazol derivative of formula (I)

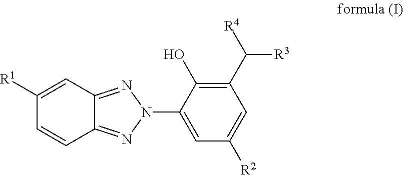

wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl; C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen.

In another aspect the invention relates to the use of a polymeric emulsifier and a benzotriazol derivative of formula (I) to reduce the whitening effect of microfine metal oxide particles.

In yet a further embodiment the invention relates to a method for reducing the whitening effect of microfine metal oxide particles, incorporated into a topical composition said method comprising the addition of a polymeric emulsifier and at least one benzotriazol of formula (I) to said composition and observing or appreciating the result (e.g. on human skin or another substrate).

The term “Cₓ-C_{y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methyl pentyl, 3-methylpentyl, 4-methylpentyl, 1,1-dimethylpropyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyle, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl, in particular C₁₋₅alkyl (mono- or poly-)substituted cyclic, bicyclic or tricyclic hydrocarbon residues such as in particular cyclopentyl, cyclohexyl, cycoheptyl or decahydronaphtyl residues. Preferably, C₅₋₁₀cycloalkyl denotes to unsubstituted or C₁₋₂alkyl (mono- or poly-)substituted cyclopentyl, cyclohexyl or cycoheptyl such as in particular to unsubstituted or methyl (mono- or poly-)substituted cyclohexyl such as most in particular cyclohexyl or 3,3,5-trimethyl-cyclohexyl.

The term “Cₓ-C_{y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term microfine metal oxide particle as used herein refers to microfine metal oxide particle suitable as inorganic UV-filter substances. Suitable metal oxides are for example titanium oxide (amorphous or crystallized in rutile and/or anatase form), iron oxide, zinc oxide, zirconium oxide or cerium oxide. For the purposes of the present invention, preferred metal oxides to be mentioned are titanium dioxide and zinc oxide, particularly titanium dioxide.

The term “microfine”, refers to a particle size from about 1 nm to 500 nm, particularly from about 10 nm to 200 nm or even 15 to 100 nm. The particles may also be coated. Such coatings are well known in the art.

Particularly preferred according to the present invention are microfine titanium dioxide particles. The particle size of the titanium dioxide is not particularly limited. All particle sizes which are principally useful for incorporating into sunscreen compositions can be used in topical compositions according to the present invention. However, the primary particle size of the titanium dioxide is usually in the range from 2 to 100 nm, preferably in the range of 5 to 50 nm and the secondary particle size is preferably in the range between 0.05 and 50 μm, preferably between 0.1 and 1 μm.

The microfine metal oxide particle may be uncoated or may be provided with a coating.

The term “coated” or “coating” means, that the metal oxide particles have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal (e.g. titanium, silicone or aluminum) alkoxides, polyethylene, silicones, proteins (collagen or elastin), alkanolamines, silicon oxides, metal oxides, sodium hexametaphosphate, alumina or glycerol. Such a surface treatment results in a coating of the metal oxide particle.

In a preferred embodiment, the metal oxide particles such as particularly the titanium dioxide are coated. Preferably the coating of the metal oxide particles comprises a silicone containing polymer and/or an inorganic oxide. More preferably, the coating comprises a silicone containing polymer, or an inorganic oxide and a silicone containing polymer. Especially preferred is a coating, which comprises an inorganic oxide and a silicone containing polymer.

The term “silicon containing polymer” refers to synthetic polymeric compounds comprising silicon atoms, which are linked via oxygen atoms to polymers and wherein the residual valences of the silicon atoms are saturated by hydrogen and/or organic residues. Examples of silicon containing polymers are silicones (also referred to as silicon oils) such as methicone, dimethicone, simethicone, polysilicone-15 or a copolymer of methicone and dimethicone.

The term “inorganic oxide” refers to oxides and hydroxides of inorganic elements, such as of silicon (e.g. silica, SiO₂), aluminum (e.g. alumina Al₂O₃, or aluminum hydroxide Al(OH)₃), zirconium or iron, preferably aluminum and silicon. Preferred inorganic oxides are alumina, aluminum hydroxide and silicon dioxide (also known as silica).

In one embodiment, the coating of the metal oxide particles comprises a silicon containing polymer and an inorganic oxide. Suitable examples include, but are not limited to coated titanium oxide particles, which were surface-treated with silica, alumina and silicone oil (such as “Micro Titanium Dioxide MT 100 SAS”, “Micro Titanium Dioxide MT 600 SAS” and “Micro Titanium Dioxide MT 500 SAS” from Tayca) or alumina and silicon (such as “UVT-M262” from Kemira). Further examples of coated titanium dioxide particles are available from BASF SE Ludwigshafen as T-Lite SF (titanium dioxide coated with aluminum hydroxide and dimethicone/methicone copolymer; titanium dioxide content 79-89 wt %), T-Lite SF-S(titanium dioxide coated with hydrated silica, dimethicone/methicone copolymer and aluminum hydroxide; titanium dioxide content 73-83 wt %) or T-lite MAX (titanium dioxide coated with dimethoxydiphenylsilane/triethoxycaprylylsilane crosspolymer, hydrated silica and aluminum hydroxide; titanium dioxide content 69-73 wt %). A further suitable coated titanium dioxide (Silica/dimethicone) is sold as PARSOL TX by DSM Nutritional Products Ltd Kaiseraugst.

Examples of coated zinc oxide particles encompass those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane), or those sold under the name “Fuji ZNO-SMS-10” by the company Fuji Pigment (ZnO coated with silica and polymethylsilsesquioxane).

In another embodiment, the coating of the metal oxide particles comprises at least one silicon containing polymer. Suitable examples include, but are not limited to titanium oxides particles treated with octylsilane (such as AEROXIDE® TiO₂ T 805 by Degussa) or with trimethoxycaprylsilane (such as Uvinul® TiO₂ by BASF SE Ludwigshafen).

Examples of zinc oxide particles coated with at least one silicon containing polymer are sold under the name “Zinc Oxide CS-5” by the company Toshibi (ZnO coated with polymethylhydrogenosiloxane), “SPD-ZI” by the company Shin-Etsu (ZnO coated with silicon-grafted acrylic polymer, dispersed in cyclodimethylsiloxane). Further coated zinc oxide particles are commercially available from BASF SE as Z-COTE HP1 (98 wt % zinc oxide and 2 wt % triethoxycaprylylsilane) or Z-COTE MAX (96-99 wt % zinc oxide and 1-4 wt % dimethoxydiphenylsilane/triethoxycaprylylsilane crosspolymer).

In another embodiment, the coating of the metal oxide particles comprises at least one inorganic oxide. Suitable examples include, but are not limited to titanium oxide particles, which were surface-treated with silica and alumina (such as “Micro Titanium Dioxide MT 500 SA” and “Micro Titanium dioxide MT 100 SA” from Tayca, and “Tioveil Fin”, “Tioveil OP”, “Tioveil MOTG” and “Tioveil IPM” from Tioxide), alumina and aluminum stearate (such as “Micro Titanium Dioxide MT 100 T” from Tayca), alumina and aluminum laurate (such as “Micro Titanium Dioxide MT 100 S” from Tayca), iron oxides and iron stearate (such as “Micro Titanium Dioxide MT 100 F” from Tayca), alumina and stearic acid (such as the product “UVT-M160” from Kemira), alumina and glycerol (such as the product “UVT-M212” from Kemira) and silicon dioxide (such as e.g. Eusolex® T-Avo and Eusolex® T-Oleo from Merck/Sachtleben).

The microfine metal oxide particles may have undergone one or at least two surface treatments.

Preferably, the microfine metal oxide particles have undergone at least two surface treatments. Thus, the particles may have a single or more coatings (muilticoating). Preferably, the microfine metal oxide particles have a multicoating such as at least a double coating. The coating may be a multicoating of an inorganic oxide such as preferably silicon dioxide (silica), and a silicon containing polymer, preferably dimethicone.

The terms “methicone [CAS 9004-73-3]” “dimethicone [CAS 63148-62-9]” and “simethicone [CAS 8050-81-5]” stand for methyl hydrogen polysiloxane, dimethyl polysiloxane, respectively, a mixture of poly(dimethylsiloxane) and silicon dioxide.

Particularly preferred microfine metal oxide particles according to the present invention are coated titanium dioxide particles in which the particles are coated by a first layer of an inorganic oxide such as preferably silica and an outer layer of an organic coating such as a silicon containing polymer (such as e.g. methicone, dimethicone or polysilicone-15), stearic acid and glycerol as well as mixtures thereof. Preferably, the inner coating is silica and the outer coating is methicone (such as e.g. the product commercialized as UV-Titan X195 by Kemira) or dimethicone (such as e.g. the product commercialized as PARSOL® TX by DSM Nutritional Products Ltd Kaiseraugst).

As used herein, the term “polymeric emulsifier” refers generally to a polymer having both hydrophilic and hydrophobic moieties that is capable of contributing to the formation of a stable emulsion between an oil phase and an aqueous phase. Any of a variety of suitable polymeric emulsifiers may be used according to the present invention. Such polymers are well known to a person skilled in the art.

The polymers for use as polymeric emulsifiers in the present invention may be of any suitable molecular weight. In certain embodiments of the invention, the polymeric emulsifier has a weight average molecular weight that is preferably greater than about 500,000, more preferably greater than about 250,000, and even more preferably greater than about 100,000 such as greater than 50,000. The molecular weight can easily be determined by GPC according to standard methods in the art.

Polymeric emulsifiers suitable for the present invention may comprise an associative polymer, i.e. a polymer formed from monomers such that individual repeat units are hydrophilic, such as may be formed by addition polymerization of such as acids as acrylic, methacrylic, maleic, itaconic, and the like or combinations to form copolymers thereof.

Notable commercially available polymeric emulsifiers include, but are not limited to, salt sensitive, hydrophobically modified polyacrylic acid such as Acrylates/C10-30 Alkyl Acrylate Crosspolymers which are commercially available under the tradename Pemulen® TR-1 and TR-2 by Noveon, Inc., water-soluble or water-swellable copolymers based on acrylamidoalkyl sulfonic acid and cyclic N-vinylcarboxamides commercially available under the tradename Aristoflex®AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation; homopolymer of acrylamidoalkyl sulfonic acid commercially available under the tradename Granthix APP by Grant Industries, Inc.

Other suitable polymeric emulsifiers include silicon based polymers such as polyether modified silicones and polyglycerin modified silicones. Polyether modified silicones include linear types, branched types and cross-linked polymer types while polyglycerin modified silicones include branched types and cross-linked polymer types. Such silicon based emulsifiers are e.g. commercially available under the tradename KF or KSG at Shin-Etsu such as e.g. KSG-210 (INCI Name Dimethicone/PEG-10/15 Crosspolymer, Dimethicone) or KF-6028 (INCI Name: PEG-9 Polydimethylsiloxyethyl Dimethicone).

Another class of polymeric emulsifiers include PEG (polyethyleneglycol)-free emulsifiers based on vegetable raw materials on the basis of polyglycerol esters or diesters of fatty acids also called polyglyceryl ester/diester (i.e. a polymer in which fatty acid(s) is/are bound by esterification with polyglycerin), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)].

Another group of polymeric emulsifiers include polyethyleneglycol esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particular suitable polymeric emulsifiers according to the present invention are selected from the group consisting of polyglycerol esters or diesters of fatty acids, polyether modified silicones (including linear types, branched types and cross-linked polymer types), polyglycerin modified silicones (including branched types and cross-linked polymer types) as well as polyethyleneglycol esters or diesters.

Most in particular the polymeric emulsifier according to the present invention is selected from the group consisting of (INCI Names) Polyglyceryl-4 Diisostearate/Polyhydroxystearate/Sebacate sold as Isolan GPS at Evonik, PEG-100 Stearate sold as Arlacel™ 165 (INCI: PEG-100 Stearate (and) Glyceryl Stearate) by Croda, Dimethicone/PEG-10/15 Crosspolymer, Dimethicone sold as KSG-210 at Shin-Etsu and PEG-9 Polydimethylsiloxyethyl, Dimethicone sold as KF-6028 at Shin-Etsu, as well as mixtures thereof.

The polymeric emulsifier may be used in the compositions of the present invention in an amount of at least 0.1 wt.-%, based on the total weight of the composition. Preferably, the amount of the polymeric emulsifier is selected in the range of about 0.3 to 3 wt.-%, preferably in the range of about 0.3 to 2 wt.-% and more preferably in the range of about 0.3 to 1 wt.-%, based on the total weight of the composition.

Preferably, only polymeric emulsifiers are used in the topical compositions of the present invention.

It is advantageous if no phosphate ester emulsifier such as e.g. potassium cetyl phosphate is present.

The topical compositions according to the present invention furthermore contain advantageously at least one co-surfactant. Co-surfactants, which are amphiphilic, relatively short chain molecules, are not suitable to form the emulsions, as they are not suitable to form micelles. However, the co-surfactants are able to fill up holes left by the polymeric emulsifier and thus further stabilize the emulsion.

Suitable co-surfactants encompass mono- and diglycerides and/or fatty alcohols. The co-surfactant is particularly used in the topical compositions according to the present invention when the emulsifier is selected from the group of polyethyleneglycol esters or diesters such as PEG-100 stearate.

The co-surfactant is advantageously used in an amount selected in the range of 0.1 to 10 wt.-% such as in the range of 0.5 to 5 wt.-% such as in particular in the range of 1 to 3 wt.-% or even in the range of 1 5o 2 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) and mixtures thereof.

The amount of the microfine metal oxide particle in the topical compositions according to the present invention is advantageously selected in the range of 0.5 to 50 wt.-% such as in particular in the range of 1 to 25 wt.-%, such as most in particular in the range of 1 to 10 wt.-%, based on the total weight of the composition.

The amount of the at least one benzotriazol derivative of formula (I) in the compositions according to the invention is preferable selected in the range of 1 to 20 wt.-%, such as in the range of 2 to 15 wt.-%, in particular in the range of 4 to 10 wt.-%, and most particular in the range of 4 to 8 wt.-%, based on the total weight of the composition.

In a particular embodiment of the present invention the benzotriazol derivative is selected from compounds of formula (I) wherein R¹ and R⁴ are hydrogen, R² is methyl and R³ is C₅₋₁₀alkoxy such as preferably C₆₋₁₀alkoxy, or C₆cycloalkoxy, such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124).

In another particular embodiment of the present invention the compound of formula (I) is a compound wherein R¹ and R⁴ are hydrogen, R² is methyl and R³ is undecyl (C₁₁H₂₃) which is commercially available as Tinogard® TL [INCI Name: benzotriazolyl dodecyl p-cresol; IUPAC Name, 2-(2H-benzotriazol-2-yl)-6-dodecyl-4-methyl-phenol] at BASF SE Ludwigshafen.

In another embodiment, the invention relates to topical compositions in the form of O/W emulsions comprising an oily phase dispersed in an aqueous phase in the presence of an O/W emulsifier wherein the compound of formula (I) is benzotriazolyl dodecyl p-cresol, the microfine metal oxide is a double coated titanium dioxide having an inner silica coating and an outer coating selected from the group of methicone and/or dimethicone and the O/W emulsifier is selected from the group consisting of (INCI Names) Polyglyceryl-4 Diisostearate/Polyhydroxystearate/Sebacate sold as Isolan GPS at Evonik, Glyceryl Stearate (and) PEG-100 Stearate sold as Arlacel™ 165 by Croda and Dimethicone/PEG-10/15 Crosspolymer, Dimethicone sold as KSG-210 at Shin-Etsu, as well as mixtures thereof

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z×(x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations (sun care products), such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, characterized in that a composition as defined above is applied to the said keratinous substances such as in particular to the skin.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention, are e.g. described in the CTFA Cosmetic Ingredient Handbook, Second Edition (1992) without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The following examples are provided to further illustrate the compositions and effects of the present invention. These examples are illustrative only and are not intended to limit the scope of the invention in any way.

### EXAMPLE

The compositions given in table 1 were prepared according to standard methods in the art.

TABLE 1
Emulsions

| | | 1A | 1B | 2A | 2B | 3A | 3B | 4A | 4B |
|---|---|---|---|---|---|---|---|---|---|
| Trade Name | INCI Name | wt.-% | | | | | | | |
| Phenonip | Phenoxyethanol & | 0.8 | 0.8 | 0.8 | 0.8 | — | — | — | — |
| | Methylparaben & Ethylparaben& | | | | | | | | |
| | Butylparaben, Isbutylparaben | | | | | | | | |
| Keltrol CG -T | Xanthan Gum | 0.3 | 0.3 | 0.3 | 0.3 | 0.3 | 0.3 | — | |
| Finsolv TN | C12-15 Alkyl Benzoate | 30 | 26 | 30 | 26 | 30 | 26 | 30 | 26 |
| AMPHISOL ® K | Potassium Cetyl Phosphate | 2.5 | 2.5 | — | — | — | — | — | — |
| Arlacel ® 165 | Glyceryl Stearate & PEG-100 | — | — | 5 | 5 | — | — | — | — |
| | Stearate | | | | | | | | |
| Isolan GPS | Polyglyceryl-4 Diisostearate/ | — | — | — | — | 2 | 2 | — | — |
| | polyhydroxystearate/Sebacate | | | | | | | | |
| KF-6028 | PEG-9 Polydimethylsiloxyethyl | — | — | — | — | — | — | 1 | 1 |
| | Dimethicone | | | | | | | | |
| KSG-210 | Dimethicone/PEG-10/15 | — | — | — | — | — | — | 3 | 3 |
| | Crosspolymer & Dimethicone | | | | | | | | |
| Lanette O | Cetearyl Alcohol | 1.5 | 1.5 | 1.5 | 1.5 | — | — | — | — |
| PARSOL ® TX | Titanium Dioxide & Dimethicone | 4 | 4 | 4 | 4 | 4 | 4 | 4 | 4 |
| | & Silica | | | | | | | | |
| Tinogard ® TL | Benzotriazolyl Dodecyl p-Cresol | — | 4 | — | 4 | — | 4 | — | 4 |
| Magnesiumsulfate | Magnesiumsulfate, Heptahydrat | — | — | — | — | 1 | 1 | — | — |
| Heptahydrat | | | | | | | | | |

| | | |
|---|---|---|
| Water dem. | Aqua | Ad 100 |

The whitening effect of the compositions was assessed using a panel of six persons. Each person applied 0.09 g of each of the respective emulsion on the forearm on marked circles of a diameter of 5 cm. Afterwards the whitening effect of the different samples was assessed visually and rated against each other with marks from 0 (no whitening) to 1 (significant whitening).

The results (mean value) are illustrated in the table 2 below.

TABLE 2
Whitening effect of emulsions comprising microfine titanium
dioxide particles

| Sample | Emusifier | Withening* |
|---|---|---|
| 1A (Reference) | Amphisol K (non polymeric) | 0.1 |
| 1B (Reference) | Reference | 0.5 |
| 2A (Reference) | Arlacel 165 (polymeric) | 0.6 |
| 2B | | 0 |
| 3A (Reference) | Isolan GPS (polymeric) | 0.5 |
| 3B | | 0.1 |
| 4A (Reference) | KSG-210 (polymeric) | 0.3 |
| 4B | | 0.1 |

Scale from 0 to 1

The whitening was assessed on a scale of 0 (no whitening) to 1 (significant whitening). As can be retrieved, the use of a polymeric emulsifier in combination with Benzotriazolyl Dodecyl p-Cresol significantly reduces the skin whitening effect of PARSOL® TX.

1. A topical composition comprising a polymeric emulsifier, a microfine metal oxide particle and at least one benzotriazol derivative of formula (I) 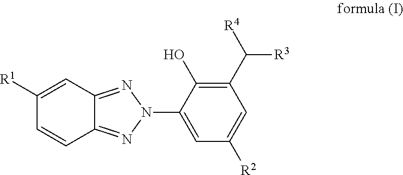
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.

2. The topical composition according to claim 1, characterized in that the benzotriazol derivative of formula (I) is present in an amount ranging from 1 to 20 wt.-%, based on the total weight of the composition.

3. The topical composition according to claim 1, characterized in that the benzotriazol derivative is used in an amount selected in the range of 2 to 15 wt.-%, based on the total weight of the composition.

4. The topical composition according to claim 1, characterized in that the microfine titanium dioxide is used in an amount selected in the range of 1 to 50 wt.-%, based on the total weight of the composition.

5. The topical composition according to claim 1 characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyloxy or undecyl.

6. The topical composition according to claim 1, characterized in that the microfine metal oxide particle is microfine titanium dioxide.

7. The topical composition according to claim 6 characterized in that the titanium dioxide is a double coated titanium dioxide having an inner inorganic silica coating and an outer organic coating selected from silicone oils, alkyl silanes, olefinic acids, stearic acid, polyols and organophosphonic acids as well as mixtures thereof.

8. The topical compositions according to claim 7, wherein the outer coating is selected from simethicone, methicone, dimethicone, polysilicone-15, stearic acid, glycerol and mixtures thereof.

9. The topical compositions according to claim 7, wherein the outer coating is selected from methicone and/or dimethicone.

10. The topical composition according to claim 1, characterized in that the polymeric emulsifier is selected from the group consisting of polyglycerol esters or diesters of fatty acids, polyether modified silicones, polyglycerin modified silicones and polyethyleneglycol esters or diesters as well as mixtures thereof.

11. The topical composition according to claim 10, characterized in that the polymeric emulsifier is selected from Polyglyceryl-4 Diisostearate/Polyhydroxystearate/Sebacate, PEG-100 Stearate, Dimethicone/PEG-10/15 Crosspolymer (and) Dimethicone, PEG-9 Polydimethylsiloxyethyl Dimethicone as well as mixtures thereof.

12. The topical composition according to claim 1, characterized in that the composition comprises at least one co-surfactant in an amount selected in the range of 0.1 to 10 wt.-%, based on the total weight of the composition.

13. The topical composition according to claim 12, characterized in that the co-surfactant is selected from the group consisting of cetyl alcohol, cetearyl alcohol, stearyl alcohol, behenyl alcohol, glyceryl stearate, glyceryl myristate, hydrogenated coco-glycerides and mixtures thereof.

14. Use of a polymeric emulsifier and a benzotriazol derivative of formula (I) 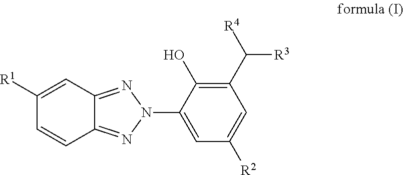
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen;
to reduce the whitening effect of microfine metal oxide particles.

15. Method for reducing the whitening effect of microfine metal oxide particles incorporated into a topical composition said method comprising the addition of a polymeric emulsifier and at least one benzotriazol of formula (I) 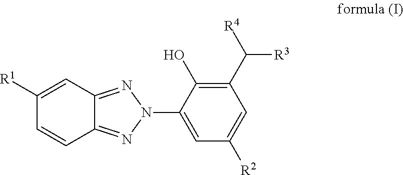
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen
into said composition and observing or appreciating the result.
